(19) United States
(12) Patent Application Publication
(10) Pub. No.: US 2006/0134055 A1
(43) Pub. Date: Jun. 22, 2006

(54) Clobetasol propionate shampoos for the treatment of seborrheic dermatitis of the scalp

(75) Inventors: Bistuer Florence, Nice (FR); Christian Loesche, Valbonne (FR); Pascale Soto, Antibes (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Galderma S.A., Cham (CH)

(21) Appl. No.: 11/017,665

(22) Filed: Dec. 22, 2004

(51) Int. Cl. A61K 8/46; C11D 1/62

(52) U.S. Cl. 424/70.24; 424/70.28

(57) ABSTRACT

Seborrheic dermatitis is effectively/safely treated by topically applying a corticosteroid shampoo, notably a clobetasol propionate shampoo, onto the scalp of a human subject afflicted therewith.

### BACKGROUND OF THE INVENTION

Seborrheic dermatitis (SD) is a common inflammation of the skin, generally occurring on the face, scalp and chest. See Gupta A K, et al., J. Eur Acad Dermatol Venereol 2004; 18(1):13-26; and Kligman A M, et al., J. Cosmetic Chemists 1976; 27:111-39. Symptoms of the disease include hyperseborrhae, dandruff, erythema, and itching. In some patients, flexural areas may also be involved. The exact role of malassezia yeasts including Malassezia furfur, formerly Pityrosporum ovale in SD pathophysiology (an abnormal host response and an inflammatory response to toxins) remains unclear. See Bergbrant I M, et al., Acta Derm Venereol 1989; 69:332-335; and Parry M E, Sharpe G R. Seborrheic dermatitis is not caused by an altered immune response to Malassezia yeast. (Br J. Dermatol 1998; 139:254-63).

Known for their excellent efficacy and anti-inflammatory profile, corticosteroids have been used for many years to treat SD. However, due to safety concerns they are being more and more replaced by antifungals such as ketoconazole. In addition, some studies demonstrated that ketoconazole was at least as effective as hydrocortisone 1% cream in the global reduction of symptoms when applied once daily. See Peter R U, et al., Br J. Dermatol 1995; 132:441-5; and Stratigos J D et al., J. Am. Acad. Dermatol 1988; 19: 850-3.

Accordingly, there is a need to develop an effective and safe method of treating SD using a corticosteroid.

### SUMMARY OF THE INVENTION

The present invention features an effective and safe treatment of seborrheic dermatitis by the application of a corticosteroid, clobetasol propionate, onto the scalp of human subjects afflicted with seborrheic dermatitis.

Specifically, the present regime or regimen is effective and safe compared with the use of ketoconazole 2% foam, and a placebo containing no active, in subjects afflicted with seborrheic dermatitis.

Accordingly, the present invention is directed to a regime or regimen for treating a human suffering from seborrheic dermatitis, comprising the steps of:

a) applying a shampoo comprising an effective amount of corticosteroid onto the scalp of the human; and

b) rinsing the scalp to remove the shampoo in a predetermined period of time after application of the shampoo of not less than two and half minutes and not more than 15 minutes.

The corticosteroid may be chosen amongst alclometasone dipropionate amcinonide, beclomethasone dipropionate, betamethasone benzoate, betamethasone dipropionate, betamethasone valerate, budesonide, clobetasol propionate, in particular, clobetasol 17-propionate, clobetasol butyrate, desonide, desoximetasone, dexamethasone, diflorasone diacetate, diflucortolone valerate, flurandrenolone, fluprednidene acetate, fluocortolone, fluocortine butyl, fluocinonide, fluocinolone acetonide, fluclorolone acetonide, flumetasone pyvalate, feudiline chlorhydrate, flumetholone, halcinonide, hydrocortisone, hydrocortisone acetate, hydrocortisone butyrate, hydrocortisone valerate, methylprednisolone acetate, mometasone furoate, methylprednisolone, prednisolone, triamcinolone acetonide, especially betamethasone salts and clobetasol propionate.

This period of time after the application of the shampoo and before the rinsing off of the shampoo from the scalp may preferably be about two and half minutes, five minutes, ten minutes or 15 minutes.

The concentration of clobetasol propionate is preferably about 0.05% of the shampoo. The shampoo may further comprise at least one surfactant and/or an alcohol. The shampoo may preferably comprise at least one of the following compounds: alcohol, coco-betaine, sodium laureth sulfate, polyquaternium, and citric acid or salt thereof.

### DETAILED DESCRIPTION OF BEST MODE AND SPECIFIC/PREFERRED EMBODIMENTS OF THE INVENTION

Study Design:

Multicentre randomized, investigator-blind, vehicle- and active-controlled, parallel group study.

Subject selection:

Male or female subjects aged from 18 to 70 years afflicted with scalp SD, defined as a total severity score (TSS, sum of erythema, loose and adherent desquamation) of at least 2;

Subjects using topical or systemic anti-SD therapies were to respect treatment specific washout periods.

Treatment:

Subjects were randomized to receive either:
0.05% clobetasol propionate shampoo to be applied for 2.5 or 5 or 10 minutes (min);
or clobetasol propionate vehicle for 10 minutes;
or ketoconazole, 2% foam for 5 minutes.

All subjects were asked to rinse off treatment after specified application time.

Products were applied twice weekly for 4 weeks.

Specifically, the 0.05% clobetasol propionate shampoo that was used in the study has the formulation described in Table 2.

Efficacy evaluation:

Score assessments at each visit included: desquamation (loose and adherent) and erythema on each quarter of the scalp. Signs were evaluated at each visit using a seven-point scale from 0 to 3, with half points being allowed;

TSS and a mean score for each sign were calculated for the whole scalp;

Other criteria were itching, as assessed at each visit by the subject on a 100 mm analogue scale and global improvement, as assessed by the investigator on a seven-point scale (from −1: worse than baseline to 5: clear) at each visit following baseline.

Safety evaluation:

Overall safety was assessed throughout based on adverse event reporting.

Results:

Subjects studied:

A total of 55 subjects (11 in each treatment group) were randomized into the study;

Four subjects withdrew from the study: one in the clobetasol propionate 10 minute group, 1 in clobetasol 5 minute, both for administrational reasons, and 2 in the clobetasol vehicle group (1 upon subject's request and one other due to lack of efficacy);

54.5% were male and 45.5% were female. The proportion of male and female subjects was similar in each treatment group except in the clobetasol 10 minute group which comprised more than 80% males;

All treatment groups were comparable in terms of race, and age (Table 1).

Efficacy:

At end point there was a statistically significant difference (all p≦0.02) for the mean TSS between the active treatments groups (0.7; 0.6; 0.8; 0.7 for clobetasol propionate 10 minute, 5 minute, 2.5 minute and ketoconazole, respectively) and the clobetasol vehicle group (2.6).

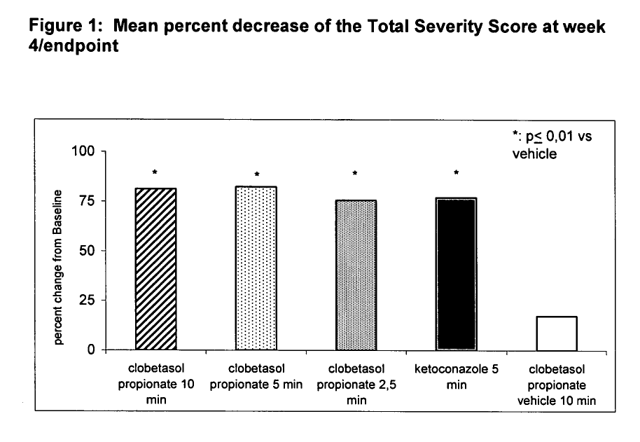

At endpoint mean percent changes for the TSS from baseline ranged from 75.6% for the 2.5 minute application to 82.3% for the 5 minute application of clobetasol and reached 76.9% for ketoconazole and 17.4% for the vehicle (FIG. 1). Differences were statistically significant with a p-value not exceeding 0.01.

Differences for mean erythema scores between the vehicle (0.7) and the active treatments were statistically significant for clobetasol propionate 5 minute (0.1; p=0.024) and ketoconazole (0.1; p=0.027).

For loose desquamation the difference to the vehicle (1.0) was statistically significant for clobetasol propionate 10 minute (0.3; p=0.027). A trend to significance could be observed for clobetasol 5 minute (0.4; p=0.051). It is of importance to note that no statistical difference could be found between ketoconazole and the vehicle on this criterion.

For adherent desquamation a statistically significant difference to the vehicle (0.9) could be shown for clobetasol propionate 5 minute (0.1; p=0.047).

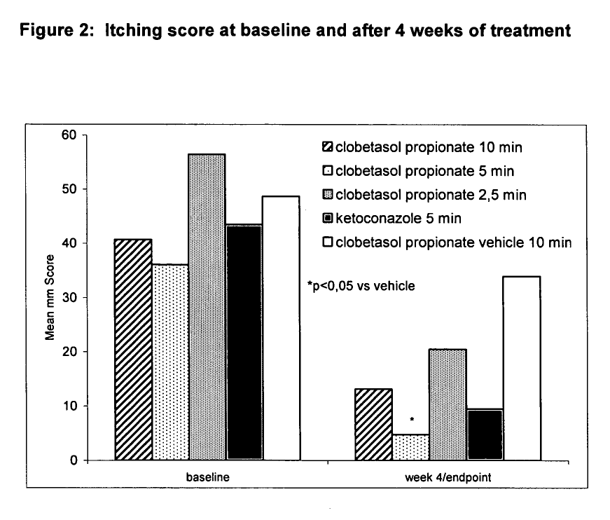

At end point, the mean score of itching (as expressed in mm) had decreased from baseline in all treatment groups. The difference between the vehicle score (34) and the active treatments scores was statistically significant for clobetasol propionate 5 minute achieving a score of 4.8 (p=0.007), FIG. 2. No statistical difference could be observed between ketoconazole and vehicle.

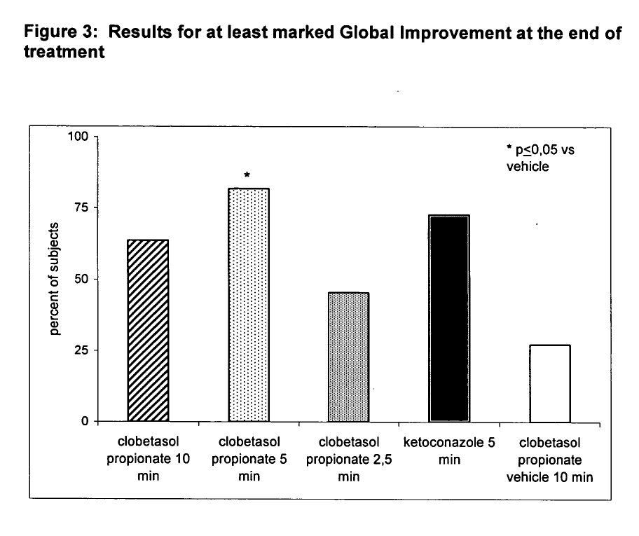

The percentage of subjects with at least marked global improvement at the end point was higher in the active treatment groups (63.7%, 81.9%, 45.5% in the clobetasol propionate 10, 5, and 2.5 minute groups, respectively, and 72.8% in the ketoconazole group) than in the clobetasol propionate vehicle group (27.3%), FIG. 3.

Clearance of SD was achieved in 45.5% of clobetasol 10 minute-treated subjects, this percentage was higher than with the other active treatments and the vehicle 9.1% for ketoconazole and the vehicle, 18.2% for clobetasol propionate 5 and 2.5 minutes). The difference between the clobetasol vehicle and the 4 active treatments was statistically significant (p-values≦0.05).

Safety:

A total of 14 adverse events were reported during the study; one subject in the 10 minute clobetasol propionate group reported dry skin considered by the investigator related to the treatment; for 1 subject treatment related folliculitis with the 5 minute clobetasol propionate application was reported. Treatment related eczema was reported for 1 subject treated with the vehicle.

There were no cases of HPA axis suppression, telangiectasia or skin atrophy reported during the course of the study.

### CONCLUSION

Accordingly, despite recent investigations suggesting that Malassezia is the causal organism of the disease and that an anti-fungal treatment is the most appropriate treatment, the present invention provides a safe and effective method of treating seborrheic dermatitis of the scalp comprising a short contact application to the scalp of a clobetasol propionate containing shampoo.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

TABLE 1
BASELINE CHARACTERISTICS

| | Clobetasol | Clobetasol | Clobetasol | | Clobetasol | |
|---|---|---|---|---|---|---|
| | propionate | propionate | propionate | Ketoconazole | propionate | |
| Demography | 10 min | 5 min | 2.5 min | 5 min | vehicle | Total |
| Number | N = 11 (%) | N = 11 (%) | N = 11 (%) | N = 11 (%) | N = 11 (%) | N = 55 (%) |
| of Subjects | | | | | | |
| Age (Years) | | | | | | |
| Mean ± SD | 39.7 ± 13.2 | 36.8 ± 13.3 | 35.5 ± 15.5 | 35.6 ± 8.4 | 37.0 ± 10.5 | 36.9 ± 12.1 |
| Minimum | 20.0 | 18.0 | 20.0 | 25.0 | 23.0 | 18.0 |
| Maximum | 63.0 | 58.0 | 64.0 | 46.0 | 53.0 | 64.0 |
| Gender | | | | | | |
| Male | 9 (81.9%) | 5 (45.5%) | 5 (45.5%) | 5 (45.5%) | 6 (54.5%) | 30 (54.5%) |
| Female | 2 (18.2%) | 6 (54.5%) | 6 (54.5%) | 6 (54.5%) | 5 (45.5%) | 25 (45.5%) |
| Race | | | | | | |
| White/Caucasoid | 11 (100%) | 11 (100%) | 11 (100%) | 10 (90.9%) | 11 (100%) | 54 (98.2%) |
| Other or mixed | — | — | — | 1 (9.1%) | — | 1 (1.8%) |
| Erythema | | | | | | |
| Mean ± SD | 1.5 ± 0.6 | 0.9 ± 0.4 | 1.0 ± 0.4 | 1.0 ± 0.7 | 1.0 ± 0.4 | 1.1 ± 0.5 |
| Loose | | | | | | |
| desquamation | | | | | | |
| Mean ± SD | 1.4 ± 0.6 | 1.2 ± 0.7 | 1.4 ± 0.7 | 1.4 ± 0.5 | 1.3 ± 0.5 | 1.4 ± 0.6 |
| Adherent | | | | | | |
| desquamation | | | | | | |
| Mean ± SD | 1.2 ± 0.6 | 1.0 ± 0.6 | 1.1 ± 0.5 | 1.0 ± 0.4 | 1.1 ± 0.5 | 1.1 ± 0.5 |
| TSS | | | | | | |
| Mean ± SD | 4.1 ± 1.4 | 3.0 ± 1.5 | 3.6 ± 1.1 | 3.4 ± 0.9 | 3.4 ± 0.8 | 3.5 ± 1.2 |
| Itching scale | | | | | | |
| Mean ± SD | 40.7 ± 27.7 | 36.1 ± 15.2 | 56.5 ± 26.2 | 43.5 ± 20.1 | 48.7 ± 20.8 | 45.1 ± 22.8 |

TABLE 2
Formulation ID No. 662.066

| Ingredient¹ | Percent (w/w) | per gram | |
|---|---|---|---|
| Clobetasol propionate, USP | 0.05% | 0.5 | mg |
| Alcohol (Ethanol 95%-96%), USP | 10.0% | 100 | mg |
| Coco-betaine (30%) | 6.0% | 60 | mg |
| Sodium laureth sulfate (70%) | 17.0% | 170 | mg |
| Polyquaternium-10 | 2.0% | 20 | mg |
| Sodium citrate dihydrate, USP | 2.6% | 26 | mg |
| Citric acid monohydrate, USP | 0.24% | 2.4 | mg |
| Purified water, USP | 62.11% | 621.1 | mg |

¹The formulation identification code number 662.066 is a GALDERMA Laboratories development code. This formulation ID code appears in other documents and sections comprising this application and is considered as the principal ID code for the formulation.

What is claimed is:

1. A regime or regimen for treating a human suffering from seborrheic dermatitis comprising the steps of:
a) applying a shampoo which comprises a thus effective amount of corticosteroid onto the scalp of the human; and
b) rinsing the scalp to remove the shampoo in a predetermined period of time after application of the shampoo of not less than two and half minutes and not more than 15 minutes.

2. A regime or regimen for treating a human suffering from seborrheic dermatitis, comprising the steps of:
a) applying a shampoo which comprises a thus effective amount of clobetasol propionate onto the scalp of the human; and
b) rinsing the scalp to remove the shampoo in a predetermined period of time after application of the shampoo of not less than two and half minutes and not more than 15 minutes.

3. The regime or regimen as defined by claim 2, wherein the concentration of clobetasol propionate is about 0.05% of the shampoo.

4. The regime or regimen as defined by claims 1 or 2, wherein the shampoo further comprises a t least one surfactant.

5. The regime or regimen as defined by claim 4, wherein the shampoo further comprises alcohol.

6. The regime or regimen as defined by claims 1 or 2, wherein the shampoo further comprises at least one of the compounds selected from the group consisting of ethanol, coco-betaine, sodium laureth sulfate, polyquaternium, and citric acid or salt thereof.

7. The regime or regimen as defined by claims 1 or 2, wherein the scalp is rinsed at about two and half minutes after the application of the shampoo onto the scalp.

8. The regime or regimen as defined by claims 1 or 2, wherein the scalp is rinsed at about five minutes after the application of the shampoo onto the scalp.

9. The regime or regimen as defined by claims 1 or 2, wherein the scalp is rinsed at about ten minutes after the application of the shampoo onto the scalp.

10. The regime or regimen as defined by claims 1 or 2, wherein the scalp is rinsed at about fifteen minutes after the application of the shampoo onto the scalp, the scalp being dry or humid.
